(19) United States
(12) Patent Application Publication
(10) Pub. No.: US 2014/0277507 A1
(43) Pub. Date: Sep. 18, 2014

(54) DISTRACTIBLE SKID SPINAL IMPLANT

(71) Applicant: Atlas Spine, Inc., Jupiter, FL (US)

(72) Inventor: Matthew G. Baynham, Jupiter, FL (US)

(73) Assignee: Atlas Spine, Inc., Jupiter, FL (US)

(21) Appl. No.: 14/214,487

(22) Filed: Mar. 14, 2014

Related U.S. Application Data
(60) Provisional application No. 61/801,102, filed on Mar. 15, 2013.

(51) Int. Cl. A61F 2/44

(52) U.S. Cl. CPC A61F 2/442
USPC 623/17.16

(57) ABSTRACT

A spinal fusion implant for implantation between adjacent vertebrae is described. The distractible spinal implant device contains a main body, a first end plate, and a second end plate. Each of the end plates are adapted to independently and slidably engagement with the main body to form a cage structure for spacing of adjacent vertebra.

### PRIORITY CLAIM

In accordance with 37 C.F.R. 1.76, a claim of priority is included in an Application Data Sheet filed concurrently herewith. Accordingly, this application is based upon and claims the priority date of U.S. Provisional Patent Application Ser. No. 61/801,102 filed Mar. 15, 2013 and entitled “Distractible Skid Spinal Implant,” the contents of which is incorporated herein by reference.

### FIELD OF THE INVENTION

This invention relates to the field of orthopedic surgery and, more particularly, to implants to be placed between vertebrae in the spine.

### BACKGROUND OF THE INVENTION

Spinal stabilization is one approach to alleviating chronic back pain caused by displaced disk material or excessive movement of individual vertebrae. Conventional stabilization techniques include fusing two or more vertebrae together to circumvent or immobilize the area of excessive movement. Normally, the vertebral disk material which separates the vertebrae is removed and bone graft material is inserted in the space for interbody fusion. In addition to, or in place of, the bone graft material, a spinal implant may be inserted in the intervertebral space.

The conventional surgical approach for stabilization has been posteriorly for ease of access to the spine and to avoid interfering with internal organs and tissue. Usually, the implant site is prepared to maintain natural lordosis and to accept a certain sized implant within certain pressure limits. This requires considerable time and skill by the surgeon.

U.S. Pat. No. 7,211,112 discloses a spinal fusion device for posterior lumbar implantation. The device employs a main body with upper and lower sections movable along an inclined ramp.

U.S. Pat. No. 6,562,074 to Gerber et al, issued May 13, 2003, discloses a spinal insert which can be manipulated to adjust the height of the implant through links connected to the upper and lower plates.

U.S. Pat. No. 6,120,506, issued Sep. 19, 2000, to Kohrs et al, discloses a lordotic implant and a tap for use in preparing the vertebrae. The implant is designed to be inserted between the non-parallel end plates of adjacent vertebrae and maintain the natural lordotic angle of the spine. This is done through the use of a threaded tapered plug inserted in a tapped hole in the direction required by the lordosis of the spine. The implant is hollow and has radial apertures for accommodating bone graft material.

U.S. Pat. No. 6,015,436, issued Jan. 18, 2000, to Shoenhoeffer, discloses a tubular spinal implant. The implant is hollow and has radial apertures for interbody fusion through bone growth material. The device is placed between adjacent vertebrae with the opposite ends of the tube contacting the opposing vertebrae. The opposite ends are threaded together to form the hollow tube.

U.S. Pat. No. 5,865,848 discloses a spinal fusion implant having two components that are complementary such that when placed together they form a whole.

Disclosed is an improved distractible implant for ease of installation.

### SUMMARY OF THE INVENTION

The present invention describes a distractible spinal implant device for insertion between two adjacent vertebrae to replace damaged, missing, or excised disk material. The distractible spinal implant device contains a cage, a first skid like end plate, and a second skid-like end plate. Each of the end plates are independently and slidably engagable with the cage.

Accordingly, it is an objective of the instant invention to teach an improved spinal implant device for insertion between two adjacent vertebrae.

It is a further objective of the instant invention to teach a distractible spinal implant device for insertion between two adjacent vertebrae.

It is yet another objective of the instant invention to teach a distractible spinal implant device for insertion between two adjacent vertebrae which contains independently movable end plates.

It is a still further objective of the invention to teach a distractible spinal implant device for insertion between two adjacent vertebrae which contains end plates which independently, slidably engage a spinal cage.

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a perspective view of an illustrative example of a distractible implant device;

FIG. 2 is a perspective view of the distractible implant device illustrating the slidable engagement of the first and second end plates with the cage;

FIG. 3 is an exploded view of FIG. 1;

FIG. 4 is a view of the plates separated from the main body;

FIG. 5 is an alternative embodiment of the end plates;

FIG. 6 is an exploded view of FIG. 5;

FIG. 7 is a pictorial view of the distractible implant device being inserted between two vertebrae;

FIG. 8 is a pictorial view of the distractible implant plates for insertion between the two vertebrae;

FIG. 9 is a pictorial view of the distractible main body for insertion between the two vertebrae; and

FIG. 10 is a pictorial view of the distractible implant device with offset insertion between misaligned vertebrae.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

Referring to the FIGS. 1-4, a distractible spinal implant device for insertion between two adjacent vertebrae to replace damaged, missing, or excised disk material, referred to generally as 10, is illustrated. The distractible spinal implant device 10 contains a cage 12, a first skid like end plate 14, and a second skid-like end plate 16. The cage 12 contains a pair of opposing side walls 18 and 20 separated by a main body 22, illustrated as a generally rectangular structure. The main body contains an aperture 24 which provides for a graft space 26. If required, the graft space can be filled with bone or bone graft material, such as bone morphogenic protein (BMP). The side walls 18 and 20 extend above and below the main body 22 and each have an inward curve 28 which form a channel 30. The channel 30 runs the length of the main body 22 and is sized and shaped to slidably engage at least a portion of the first skid like end plate 14 and the second skid-like end plate 16. Portions of the side walls 18 and 20 may contain tapered surfaces 32 to aid with insertion.

The first skid like end plate 14 contains a first end 34, a second end 36, and a first end plate elongated body 38. The first end plate elongated body 38 may contain an aperture 40 which provides for a graft space 42 and is preferably sized to the length of the cage 12. An upper surface 46 contains gripping members 48, illustrated as gripping teeth. A second lower surface 50 is preferably smooth in order to allow for slidable movement within the channel 30. At the second end 36, a fin-like or winged structure 52 extends away, in an upwardly direction from the upper surface 46. As illustrated, the fin-like or winged structure 52 is angled inwardly toward the upper surface 46, thereby forming an angled surface 54.

The second skid like end plate 16 contains the same structures as the first skid like end plate 14 and is aligned with the first skid like end plate 14 in a parallel, but reverse orientation. Accordingly, the second endplate 16 contains a first end 56, a second end 58, and a second end plate elongated body 60. The second end plate elongated body 60 may contain an aperture 62 which provides for the graft space 42 and is preferably sized to the length of the cage 12. An upper surface 64 contains gripping members 66, illustrated as gripping teeth. A second lower surface 68 is preferably smooth in order to allow for slidable movement within the channel 30. At the second end 58, a fin-like or winged structure 70 extends away, in an upwardly direction from the upper surface 64. As illustrated, the fin-like or winged structure 70 is angled inwardly toward the upper surface 64, thereby forming an angled surface 72. When aligned with the first end plate 14, the fin-like or winged structures 52 and 70 form an arrow like structure. Fin-like or winged structures 52 and 70 may be formed as separate units secured to the first or second end plate, using, for example, welding techniques, or may be integrally formed as single units. One or both of the end plates 14 or 16 may also contain fixation or locking barbs 74.

Referring to FIGS. 5 and 6, set forth is an alternative embodiment. The distractible spinal implant device 100 contains a cage 112, a first skid like end plate 114, and a second skid-like end plate 116. The cage 112 contains a pair of opposing side walls 118 and 120 separated by a main body 122, illustrated as a generally rectangular structure. The main body contains an aperture 124 which provides for a graft space 126. If required, the graft space can be filled with bone or bone graft material, such as bone morphogenic protein (BMP). The side walls 118 and 120 extend above and below the main body 122 forming a channel that runs the length of the main body and is sized and shaped to slidably engage the first skid like end plate 114 along the upper surface and the second skid-like end plate 116 along the lower surface. Portions of the side walls 18 and 20 may contain tapered surfaces 132 to aid with insertion.

The first skid like end plate 114 contains a first end 134, a second end 136, and a first end plate elongated body 138. The first end plate elongated body 138 may contain an aperture 140. An upper surface 146 contains gripping members 148, illustrated as gripping teeth. A second lower surface 150 includes teeth for engagement of the upper surface of the main body. At the second end 136, a fin-like or winged structure 152 extends away, in an upwardly direction from the upper surface 146. As illustrated, the fin-like or winged structure 152 is angled inwardly toward the upper surface 146, thereby forming an angled surface 154.

The second skid like end plate 116 contains the same structures as the first skid like end plate 114 and is aligned with the first skid like end plate 114 in a parallel, but reverse orientation. Accordingly, the second end plate 116 contains a first end 156, a second end 158, and a second end plate elongated body 160. The second end plate elongated body 160 may contain an aperture 162 which provides for graft space. An upper surface 164 contains gripping members 166, illustrated as gripping teeth. A second lower surface 168 includes teeth to engage teeth 167 between the channel formed on the main body. At the second end 158, a fin-like or winged structure 170 extends away, in an upwardly direction from the upper surface 164. As illustrated, the fin-like or winged structure 170 is angled outwardly thereby forming an angled surface 172. When aligned with the first end plate 114, the fin-like or winged structures 152 and 170 form an arrow like structure. Fin-like or winged structures 152 and 170 may be formed as separate units secured to the first or second end plate, using, for example, welding techniques, or may be integrally formed as single units. One or both of the end plates 114 or 116 may also contain fixation or locking barbs 174.

Referring now to FIGS. 7-9, the spinal implant device is inserted between two adjacent vertebrae 202 and 204 using an insertion instrument 200 to replace damaged, missing, or excised disk material and to act as a distracter by increasing the space between vertebrae. The first and second end plates 14 and 16 are inserted into the disc space by attachment to the end of the tongs 206 and 208 of the insertion instrument. Once properly oriented, the main body 22 is inserted in between the plates 14 and 16 by sliding along the tongs 206 and 208 by pushing the handle 210 secured to the main body 22. In this embodiment, the plates 14 and 16 engage the channels formed in the main body 22. In the alternative embodiment, the channels are open and the plates 114 and 116 are maintained with the main body 122 by compression of the adjacent vertebrae 202 and 204. The fin-like or winged structures act as a buttress stop.

Because each end plate 14 and 16 is independently engageable and slidable with the cage 12, the spinal implant device 10 may be used for vertebrae that are misaligned. Similarly, the alternative embodiment employs plate 114 and 116 which are independently engageable and slidable with the cage 112, wherein the spinal implant device may be used for vertebrae that are misaligned. Referring to FIG. 10, two misaligned vertebrae 202 and 204 are illustrated. The spinal implant is shown with the first end plate 14 slid out to engage the first vertebra 202, while the second end plate does not require the same amount of extension to engage the second vertebra 204.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A distractible skid spinal implant comprising: a substantially rectangular main body defined by an upper surface spaced apart from a lower surface with a first sidewall having an upper section extending above the upper surface of the main body and a lower section extending below the lower surface of the main body, and a second sidewall having an upper section extending above the upper surface of the main body and a lower section extending below the lower surface of the main body, said second sidewall forming a mirror image of said first sidewall;
a first end plate constructed and arranged for placement between said upper sections above the upper surface of the main body; and a second end plate independent of said first end plate constructed and arranged for placement between said lower sections of said lower surface of the main body;
wherein said first and second end plates are positioned between two adjacent vertebra and said main body placed therebetween to allow spacing of the adjacent vertebra.

2. The spinal implant according to claim 1 wherein said first and second end plate each include inner teeth for engaging said main body and gripping teeth placed along an outer surface for engaging bone.

3. The spinal implant according to claim 2 wherein said inner teeth and said gripping teeth are directional to allow ease of insertion of the main body between the plates and inhibit retraction.

4. The spinal implant according to claim 2 wherein said first and second end plates include an end wall extending outwardly from said outer surface a distance to act as a buttress stop.

5. The spinal implant according to claim 2 wherein said first and second end plates include a centrally disposed aperture for passage of bone growth material.

6. The spinal fusion implant according to claim 1 wherein said main body includes a centrally disposed aperture for receipt of bone growth material.

7. A distractible skid spinal implant comprising: a substantially rectangular main body defined by an upper surface spaced apart from a lower surface with a first sidewall having an upper section extending above the upper surface of the main body with an inward curve forming an upper channel and a lower section extending below the lower surface of the main body with an inward curve forming a lower channel, and a second sidewall having an upper section extending above the upper surface of the main body with an inward curve forming an upper channel and a lower section extending below the lower surface of the main body with an inward curve forming a lower channel, said second sidewall forming a mirror image of said first sidewall;
a first end plate constructed and arranged to slidably engage the upper channels of said first and second sidewall; and
a second end plate independent of said first end plate constructed and arranged to slidably engage said lower channels of said first and second sidewall;
wherein said first and second end plates are positioned between two adjacent vertebra and said main body placed therebetween to allow spacing of the adjacent vertebra.

8. The spinal implant according to claim 7 wherein said first and second end plate each include a smooth inner surface for slidable movement over said main body and gripping teeth placed along an outer surface for engaging bone.

9. The spinal implant according to claim 8 wherein said teeth are directional to allow ease of insertion of the main body between the end plates and inhibit retraction by engaging bone structure.

10. The spinal implant according to claim 7 wherein said first and second end plate include an end wall extending outwardly from said outer surface of a distance to act as a buttress stop.

11. The spinal implant according to claim 7 wherein said first and second end plates include a centrally disposed aperture for passage of bone growth material.

12. The spinal fusion implant according to claim 7 wherein said main body includes a centrally disposed aperture for receipt of bone growth material.
